(19) United States
(12) Patent Application Publication
(10) Pub. No.: US 2023/0087196 A1
(43) Pub. Date: Mar. 23, 2023

(54) Application of circIKBKB Inhibitors and Test Reagents thereof in Diagnosis, Treatment and Prognosis Kits for Breast Cancer Bone Metastasis

(71) Applicants: SUN YAT-SEN UNIVERSITY CANCER CENTER, Guangzhou (CN); SUN YAT-SEN UNIVERSITY, Guangzhou (CN)

(72) Inventors: Jun LI, Guangzhou (CN); Libing Song, Guangzhou (CN); Yingru XU, Guangzhou (CN); Shuxia ZHANG, Guangzhou (CN); Xingui WU, Guangzhou (CN); Xinyi LIAO, Guangzhou (CN); Man LI, Guangzhou (CN); Suwen CHEN, Guangzhou (CN); Xincheng LI, Guangzhou (CN)

(21) Appl. No.: 17/817,650

(22) Filed: Aug. 4, 2022

Related U.S. Application Data
(63) Continuation-in-part of application No. PCT/CN2021/108014, filed on Jul. 23, 2021.

(30) Foreign Application Priority Data
Jul. 19, 2021 (CN) 202110812312.2

(51) Int. Cl. A61K 31/496; A61P 35/04; C12Q 1/6886

(52) U.S. Cl. CPC A61K 31/496; A61P 35/04; C12Q 1/6886; C12Q 2600/158

(57) ABSTRACT

The invention belongs to the field of biotechnology and medicine, and particularly relates to the application of an inhibitor of circIKBKB and a testing reagent thereof in diagnosis, treatment and prognosis kits of breast cancer bone metastasis. The kit includes one of RT-PCR, Q-PCR, Northern blot, FISH or ISH kits. The kit includes the primers shown in SEQ ID Nos. 1-2 in the sequence listing or the probe shown in SEQ ID NO. 3 in the sequence listing for the back-splice junction sequences for circIKBKB. An antisense oligonucleotide targeting the linker sequence of circIKBKB is used as a reagent for inhibiting the generation of circIKBKB and for the preparation of a drug for the treatment of breast cancer bone metastasis. The eIF4A3-IN-2 inhibitor is used for the preparation of a treatment for breast cancer bone metastasis drug.

### CROSS REFERENCE OF RELATED APPLICATIONS

This application is a continuation of PCT International Patent Application No. PCT/CN2021/108014, filed on 23 Jul. 2021, entitled “Application of circIKBKB Inhibitors and Test Reagents thereof in Diagnosis, Treatment and Prognosis Kits for Breast Cancer Bone Metastasis,” which claims foreign priority of Chinese Patent Applications No. 20211081232.2, filed 19 Jul. 2021 in the China National Intellectual Property Administration (CNIPA), the entire contents of which are hereby incorporated by reference in their entireties.

### REFERENCE TO AN ELECTRONIC SEQUENCE LISTING

The contents of the electronic sequence listing (5-sequence-circIKBKB-20220801-2200.xml; Size: 5,579 bytes; and Date of Creation: Aug. 1, 2022) is herein incorporated by reference in its entirety.

### FIELD OF THE INVENTION

The invention belongs to the technical field of biotechnology and medicine, and particularly relates to the application of circIKBKB inhibitors and testing reagents thereof in diagnosis, treatment and prognosis kit for breast cancer bone metastasis.

### BACKGROUND OF THE INVENTION

The International Agency for Research on Cancer (IRAC) of World Health Organization (WHO) released the latest cancer burden data in December 2020, and the incidence of breast cancer has surpassed lung cancer as the most common type of cancer in humans worldwide. In the United States, breast cancer is also the commonly diagnosed cancer in women, with a mortality rate second only to lung cancer. Most breast cancer patients have distant metastases at the time of initial diagnosis. Even in early-stage breast cancer patients, there may be a low circulating tumor burden, which will lead to tumor recurrence in the future. Bone is the most common sites of distant metastasis of breast cancer, and bone metastases are reported in 74% of all breast cancer deaths at autopsy. Therefore, no matter for early stage or advanced breast cancer, early detection of bone metastasis is of great significance for clarifying the stage of tumor metastasis, adjusting the treatment and improving the treatment prognosis of patients with tumor.

At the molecular level, breast cancer is a heterogeneous disease. Molecular features include activation of human epidermal growth factor receptor 2 (HER2, encoded by ERBB2), activation of hormone receptors (estrogen receptor and progesterone receptor), and/or BRCA mutations. Treatment strategies differ according to molecular subtype. Management of breast cancer is multidisciplinary, it includes locoregional (surgery and radiation therapy) and systemic therapy approaches. Systemic therapies include endocrine therapy for hormone receptor-positive disease, chemotherapy, anti-HER2 therapy for HER2-positive diseases, bone stabilizing agents, poly (ADP-ribose) polymerase inhibitors for BRCA mutation carriers, and quite recently immunotherapy. However, the treatment regimen for breast cancer in situ is not suitable for treating bone metastasis of breast cancer.

Metastasis is the most important biological characteristic of malignant tumors, and the most frequent site of breast cancer metastasis is the bone. Bone metastasis is associated with severe morbidities in patients with advanced breast cancer. Most patients with bone metastases experience complications, so-called skeletal-related events (SREs), including hypercalcemia, severe bone pain, pathological fractures, spinal cord compression, and bone orthopedic surgery owing to bone instability. Therefore, it is of great interest to improve the clinical management of these patients by early diagnosis and clear prognosis for patients at risk of developing skeletal complications. However, at present, various treatments of patients with breast cancer bone metastasis cannot significantly increase the median survival time.

Currently, imaging and tissue biopsy are the major means for early detection or monitoring of breast cancer bone metastasis. Nevertheless, imaging and pathological examinations are limited in terms of diagnostic accuracy and sensitivity, while the commonly used serum markers carcinoembryonic antigen (CEA) and carbohydrate antigen 153 (CA153) show a poor diagnostic ability.

Osteolytic metastasis is the most common form of breast cancer bone metastasis. At present, markers of bone resorption mainly include cross-linked N-telopeptide of type I collagen (NTX). NTX is a stable and specific final product produced by osteoclasts after dissolution of bone matrix, which can reflect the activity of osteoclasts. Several studies have confirmed the role of NTX in the diagnosis and efficacy evaluation of solid tumor bone metastasis. It was found that NTX has important reference significance in the diagnosis of bone metastasis and can assist in the timely diagnosis of bone metastasis of malignant tumors. OSTEOMARK® NTx Serum is a competitive inhibition enzyme-linked immunosorbent assay (ELISA/EIA) for quantitative determination of NTX in human serum. However, this method can only detect the level of NTX when bone metastasis is formed and osteolysis increases, so it cannot play a role in predicting bone metastasis. In addition, the level of NTX is affected by many factors, and the diagnostic reference is distributed in a wide range.

Therefore, it is crucial to find a robust method to detect early breast cancer and monitor breast cancer bone metastasis.

At present, bone-protecting agents are the commonly used clinical drugs of the treatment options in patients with breast cancer bone metastasis. The bone-protecting agents includes bisphosphonates and denosumab, receptor activator of nuclear factor κ-B ligand (RANKL) inhibitor. However, the serious side effects of these drugs pose long-term safety concerns. In addition, the currently available treatment options for metastatic breast cancer in the bones cannot significantly increase median survival time. Thus, starting from the molecular mechanism related to bone metastasis of breast cancer to find new targeted drugs is the focus of future research.

### SUMMARY OF THE INVENTION

In view of the above problems, the purpose of the invention is to provide an application of a testing reagent of circIKBKB in diagnosis, treatment and prognosis kit of breast cancer bone metastasis and an inhibitor of circIKBKB. CircIKBKB is highly expressed in patients of breast cancer bone metastasis. The kit is more characteristic and more sensitive to early predict the occurrence of breast cancer bone metastasis, diagnose breast cancer bone metastasis, predict disease progression, evaluate treatment effects, guide drug use and prognosis evaluation, and as the basis for the treatment of bone metastasis.

The technical content of the present invention is as follows:

The invention provides the application of a testing reagent for the expression level of molecular marker circIKBKB in the preparation of a diagnosis, treatment and prognosis reagent for breast cancer bone metastasis.

The circular RNA marker circIKBKB is hsa_circ_0084100.

The breast cancer includes one of ER⁺ breast cancer, HER2⁺ breast cancer, and triple negative breast cancer.

The invention also provides the application of a testing reagent for the expression level of molecular marker circIKBKB in diagnosis, treatment and prognosis kit of breast cancer bone metastasis.

The invention also provides a kit for detecting the expression level of the marker circIKBKB.

Application of the kit in the preparation of diagnostic, therapeutic and prognostic reagents for breast cancer bone metastasis;

The kit includes reagents capable of quantitatively detecting the expression level of circIKBKB.

The kit includes RT-PCR, Q-PCR, Northern blot, FISH or ISH kits.

The kit includes the primers shown in SEQ ID NOs: 1-2 in the sequence listing or the probe shown in SEQ ID NO: 3 in the sequence listing for the linker sequence of circIKBKB.

The present invention also provides an antisense oligonucleotide (ASOs) targeting the linker sequence of circIKBKB, using as a reagent for inhibiting the generation of circIKBKB and for preparing a drug for treating breast cancer bone metastasis.

The sequences of the ASOs are shown in SEQ ID NOs: 4-5 in the sequence listing.

The invention also provides an eIF4A3-IN-2 inhibitor as a reagent for inhibiting the generation of circIKBKB and for preparing a drug for treating breast cancer bone metastasis.

The eIF4A3-IN-2 is an inhibitor of EIF4A3.

The beneficial effects of the present invention are as follows:

The application of the testing reagent of the molecular marker circIKBKB expression level of the present invention in the preparation of breast cancer bone metastasis diagnosis, treatment and prognosis reagents and kits. The circIKBKB promotes breast cancer bone metastasis by promoting the differentiation and maturation of osteoclast precursors, and the circIKBKB can be highly expressed in patients of breast cancer with specific bone metastasis. It can predict the risk of bone metastasis in patients, so as to prevent the bone metastasis. In contrast, test kits in prior art can only detect bone metastasis after they have occurred. The circIKBKB detection kit is more characteristic and more sensitive to early predict the occurrence of breast cancer bone metastasis, diagnose breast cancer bone metastasis, predict disease progression, evaluate treatment effects, guide drug use and prognosis evaluation, and as a basis for the treatment of bone metastasis.

The kit for detecting the expression level of the marker circIKBKB of the present invention includes a reagent capable of quantitatively detecting the expression level of circIKBKB, and is used for the diagnosis, treatment and prognosis of breast cancer bone metastasis.

The ASOs targeting the linker sequence of circIKBKB of the present invention is used to prepare a drug for the treatment of breast cancer bone metastasis. By inhibiting the differentiation and maturation of osteoclast precursors, the bone metastasis of breast cancer can be inhibited.

The eIF4A3-IN-2 of the present invention is used for preparing a medicine for treating breast cancer bone metastasis. The inhibitor inhibits the bone metastasis of breast cancer by inhibiting the differentiation and maturation of osteoclast precursors, thereby achieving the effect of treating bone metastasis.

### BRIEF DESCRIPTION OF DRAWINGS

FIG. 1 shows the results of circRNA deep sequencing of 6 primary breast cancer tissues (without bone metastasis) and 6 bone metastatic tissues of breast cancer.

FIG. 2 shows the results of circIKBKB expression in the primary breast cancer tissues (with bone metastasis) detected by RT-PCR.

FIG. 3A shows the ISH experiment of detecting the expression of circIKBKB, and FIG. 3B is a Kaplan-Meier analysis showing the relationship between circIKBKB expression level and prognosis of bone metastasis in breast cancer.

FIG. 4 shows the results of circIKBKB expression in bone metastasis cell lines detected by Q-PCR.

FIG. 5A shows the result of studying the effect of circIKBKB overexpression on breast cancer bone metastasis by in vivo bone metastasis model monitored by bioluminescence imaging; FIG. 5B shows the result of studying the effect of circIKBKB overexpression on breast cancer bone metastasis by μCT analysis; and FIG. 5C shows the result of studying the effect of circIKBKB overexpression on breast cancer bone metastasis by TRAP staining.

FIG. 6A shows the osteoclast differentiation assay by TRAP staining (upper) or osteoblast differentiation assay by ALP staining (lower) in the presence of CM from indicated cells (left), and quantification of number of TRAP+-multinuclear osteoclasts, TRAP activity and ALP activity from experiment in left panel (right); FIG. 6B shows the phase contrast micrograph of pre-osteoclasts treated with CM from indicated cells (left) and IF staining images of phalloidin (F-actin) (middle and right), and quantification of the number of fused multinuclear cells from experiment in the left panel (right); and FIG. 6C shows the bone resorption assay analysis of pre-osteoclasts cultured onto the bone slices treated with CM from indicated cells (left), then bone slice was fixed for scanning electron microscopy (SEM) (middle) and quantification of the number of resorption pits per bone slice (right).

FIG. 7A shows the qRT-PCR analysis of mRNA level of osteoclast differentiation markers in pre-osteoclasts treated by CM from the indicated BC cells; FIG. 7B shows the schematic illustration of “vicious cycle” between cancer cells and osteoclasts (upper), and ELISA analysis of TGF-β1 levels in CM from pre-osteoclasts (lower left), and MTT analysis of growth curves of breast cancer cells from experiment in the upper panel (lower right).

FIG. 8A shows the normalized BLI signals of bone metastases and Kaplan-Meier bone-metastasis-free survival curve of mice from the indicated experimental groups (n=8/group); FIG. 8B shows the BLI, μCT (longitudinal and trabecular section) and histological (H&E and TRAP staining) images of bone lesions from representative mice (left), and quantification of circIKBKB and IKBKB expression and μCT osteolytic lesion area and TRAP+ osteoclasts along the bone-tumor interface of metastases from experiment in the left panel (right); and FIG. 8C shows the quantification of bone parameters from representative mice in FIG. 8B. BV/TV, bone/tissue volume ratio; BS/TV, bone surface/tissue volume ratio; Tb. n, trabecular number; Tb. sp., trabecular separation; Tb. th., trabecular thickness; TBPf, trabecular bone pattern factor.

FIG. 9A shows the result of TRAP staining, which shows inhibiting the osteoclastogenesis induced by cancer cells after circIKBKB ASOs treatment; FIG. 9B shows the result of phalloidin staining, which shows inhibiting the osteoclastogenesis induced by cancer cells after circIKBKB ASOs treatment; and FIG. 9C shows the result of bone resorption experiments, which shows inhibiting the bone resorption induced by cancer cells after circIKBKB ASOs treatment.

FIG. 10 shows the qRT-PCR analysis of mRNA level of osteoclast differentiation markers in pre-osteoclasts treated by CM from the indicated BC cells.

FIG. 11A shows the schematic illustration of circIKBKB pre-mRNA pull-down following the mass spectrometry to identify circIKBKB cyclization factor; FIG. 11B shows the real-time PCR analysis of circIKBKB expression in the indicated SCP2 cells; and FIG. 11C shows the real-time PCR analysis of circIKBKB and IKBKB expression in EIF4A3-overexpressing and control MDA-MB-231 cells.

FIG. 12A shows the putative binding sites of EIF4A3 in the upstream and downstream region of the circIKBKB pre-mRNA predicted with circinteractome database (left), and RIP assay analysis of interaction of EIF4A3 with circIKBKB pre-mRNA in SCP2 cells; and FIG. 12B shows the schematic diagram of 7 fragments of circIKBKB pre-mRNA (upper) and RNA pull-down assay analysis (lower) of the interaction between eIF4A3 and above 7 fragments of circIKBKB pre-mRNA.

FIG. 13 shows the image (left) and quantification (right) of TRAP+-multinuclear osteoclasts stimulated by CM from the indicated cells.

FIG. 14A shows the IHC analysis (left) and quantification (right) of EIF4A3 expression in 20 normal breast tissues and 331 clinical breast cancer tissues, including 295 primary breast cancer tissues (237 non-BM/BC and 58 BM/BC) and 36 bone-metastatic BC tissues (at bone); and FIG. 14B shows the Kaplan-Meier analysis of bone metastasis-free survival curves in patients with BM/BC with low vs high expression of EIF4A3 (n=58; P<0.001, log-rank test).

FIG. 15 shows the two representative specimens (left), and percentages of specimens showing low or high EIF4A3 expression in relative to the levels of circIKBKB (right); and FIG. 15B shows the Kaplan-Meier analysis of bone metastasis-free survival curves in patients with BM/BC with low vs high expression of EIF4A3 (n=58; P<0.001, log-rank test).

FIG. 16 shows the image of TRAP+-multinuclear osteoclasts (upper) and resorption pit (lower) treated with CM from indicated cells (left), and quantification of TRAP+-multinuclear osteoclasts, TRAP activity and resorption pits per bone slice from experiment in the left panel (right).

FIG. 17A shows the normalized BLI signals of bone metastases and Kaplan-Meier bone metastasis-free survival curve of mice from the indicated experimental groups (n=8/group); and FIG. 17B shows the BLI and μCT images (left) and histological (H&E and TRAP) images (middle) of bone lesions from mice, which received vehicle or eIF4A3-IN-2 treatment started at 2 days after intracardial injection of SCP2 cells, and quantification of osteolytic sites and TRAP+ osteoclasts along the bone-tumor interface of metastases from experiment in left and middle panel.

FIG. 18 shows the BLI and μCT images (left) and histological (H&E and TRAP) images (middle) of bone lesions from mice, which vehicle or eIF4A3-IN-2 was started when bone-metastatic tumors formed, and quantification of osteolytic sites and TRAP+ osteoclasts along the bone-tumor interface of metastases from experiment in left and middle panel (right).

### DETAILED DESCRIPTION OF THE INVENTION

The present invention will be described in further detail below through specific implementation examples and accompanying drawings. Various equivalent modifications of the invention are within the meaning of the appended claims.

Unless otherwise specified, all the raw materials and reagents of the present invention are the raw materials and reagents in the conventional market.

In the following specific implementation cases, the experimental method adopts conventional experimental conditions, or in accordance with the conditions described in the “Molecular Cloning Experiment Guide” (third edition).

### Embodiment 1

An application of a marker circIKBKB in the diagnosis, treatment and prognosis of breast cancer bone metastasis:

In the experiment to detect the expression level of circIKBKB, the detection primers used include:

circIKBKB-RT-PCR-F:
(SEQ ID NO: 1)
5′-CCAGTTTGAGAACTGCTGTGG-3′
circIKBKB-RT-PCR-R:
(SEQ ID NO: 2)
5′-CGGCACTGCTTGATGGCAA-3′.

Detection probe circIKBKB-ISH-probe:

(SEQ ID NO: 3)
5′-ATCCTCACCTTGCTGAGTGACATTGGAAACAGGTGAGCAGATTGCC
ATCA-3′.

1. Elevated expression of circIKBKB in bone metastatic tissues of breast cancer and its clinical association with the occurrence of bone metastasis:

Experiment 1: circRNA deep sequencing was performed in 6 primary breast cancer tissues without bone metastasis and 6 bone metastatic tissues of breast cancer;

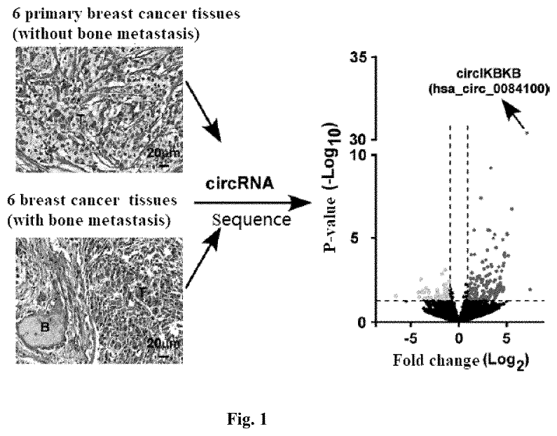

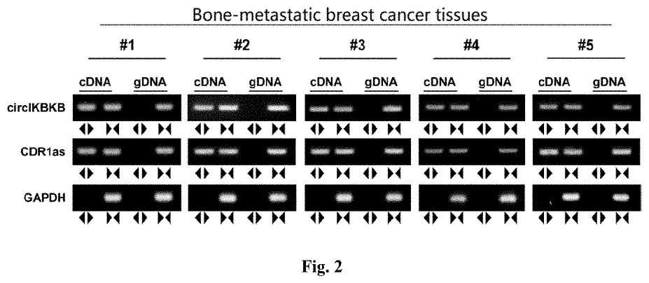

As shown in FIG. 1, a total of 214 circular RNAs were dysregulated in bone metastatic breast cancer tissue compared with primary breast cancer tissue (without bone metastasis), including 163 significantly upregulated and 51 downregulated circRNAs, among which circIKBKB was most significantly upregulated.

Experiment 2: The expression of circIKBKB was detected by reverse transcription-polymerase chain reaction (RT-PCR) in 5 cases of bone metastatic breast cancer tissues.

Experiment 3: The expression of circIKBKB was examined using in situ hybridization (ISH), with a specific probe targeting the junction sequence of circIKBKB, in 20 normal breast tissues and 331 clinical breast cancer tissues, including 295 primary breast cancer tissues (237 without bone metastasis and 58 with bone metastasis) and 36 bone-metastatic breast cancer tissues (at bone).

Experiment 4: Based on ISH results, 58 primary breast cancer tissues (with bone metastasis) were divided into two groups, high expression of circIKBKB and low expression of circIKBKB, and the correlation between the circIKBKB expression and breast cancer bone metastasis was analyzed.

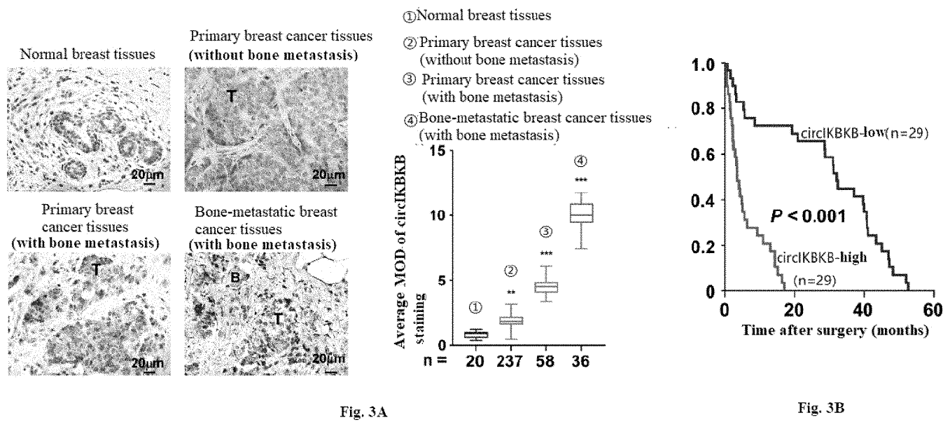

The circIKBKB signal were undetectable in normal breast tissues, and only marginally detectable in primary breast cancer tissues without bone metastasis but increased in primary breast cancer tissues with bone metastasis and were elevated markedly in bone-metastatic breast cancer tissues (as shown in FIGS. 2 and 3A). Importantly, ISH statistical analysis revealed that patients with high circIKBKB-expressing breast cancer had significantly shorter bone-metastasis-free survival than those with low circIKBKB-expressing breast cancer (as shown in FIG. 3B).

The above shows that the expression of circIKBKB is increased in the tissues of breast cancer patients with bone metastases, and is closely related to the occurrence of bone metastasis, which can be used for the diagnosis, treatment and prognosis of breast cancer bone metastasis.

2. Overexpression of circIKBKB induces osteolytic bone metastasis of breast cancer:

Experiment 1: The expression of circIKBKB was detected in 5 breast cancer cell lines by quantitative real-time reverse transcription-polymerase chain reaction (qRT-PCR).

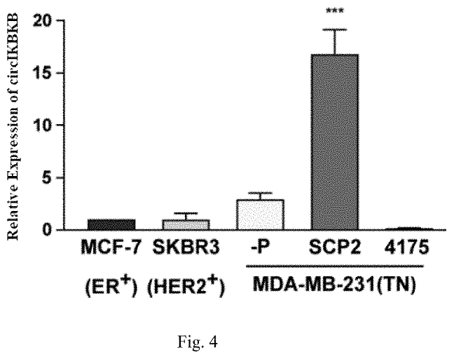

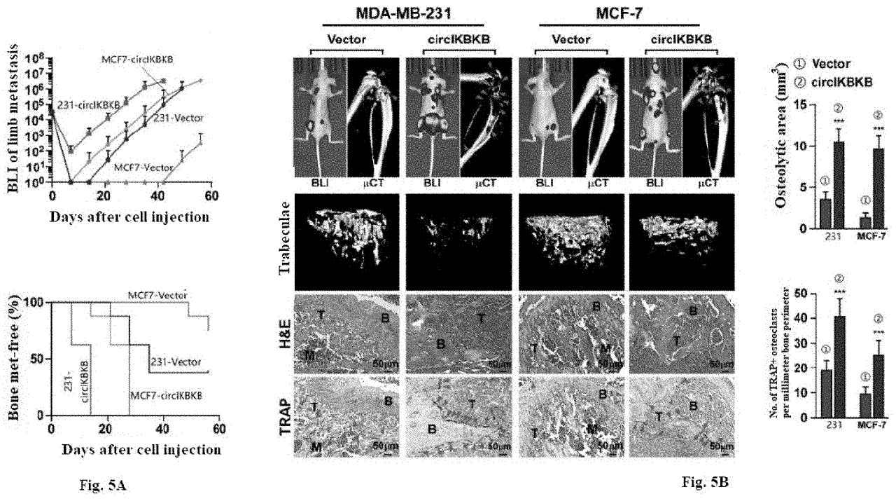

As shown in FIG. 4, circIKBKB was highly expressed in the bone-tropic breast cancer cell line SCP2 cells compared with low bone metastatic breast cancer cell lines such as MCF7, SKBR3, MDA-MB-231 and 4175.

Experiment 2: After constructing cell lines stably expressing luciferase (luci) in MCF7 and MDA-MB-231 breast cancer cells, MCF7-Vector, MCF7-circIKBKB, MDA-MB-231-Vector, MDA -MB-231-circIKBKB cells were seeded into the left ventricle of nude mice (1*10⁶ cells/mouse). The mice were sacrificed 60 days later, and hindlimbs were removed for microcomputed tomography (μCT) analysis, H&E and tartrate-resistant acid phosphatase (TRAP) staining.

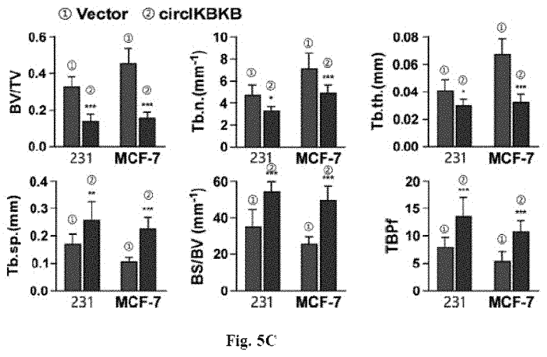

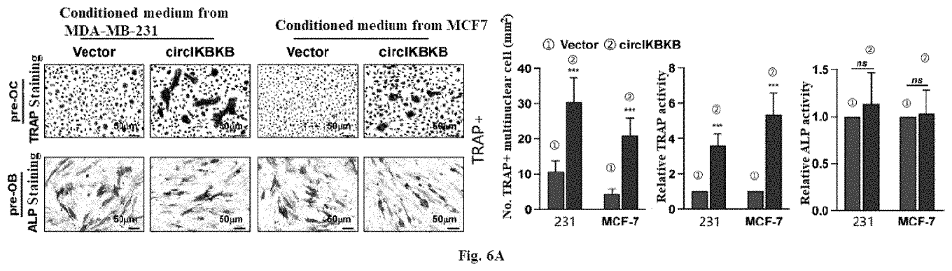

An in vivo model of bone metastasis monitored by bioluminescence imaging (BLI) revealed that mice injected intracardially with breast cancer cells overexpressing circIKBKB exhibited earlier bone metastasis development and greater bone metastatic tumor burden (as shown in FIG. 5A and FIG. 5B). μCT analysis indicated that, compared with Vector control mice, circIKBKB/mice exhibited larger osteolytic bone lesions and significantly modulated bone parameters such as reduced trabecular bone volume/number/thickness, trabecular bone separation/bone pattern factor increases (as shown in FIG. 5B ad FIG. 5C). At the same time, we observed a significant increase in the number of TRAP+ osteoclasts along the bone-tumor interface in circIKBKB/mice (FIG. 5B). This suggests that circIKBKB-overexpressing breast cancer cells might possess a strong capability to induce the formation of bone pre-metastatic niche.

Experiment 3: Osteoclast precursors and osteoblast precursors were treated with cell supernatants of MCF7-Vector, MCF7-circIKBKB, MDA-MB-231-Vector, MDA-MB-231-circIKBKB, and then via staining by TRAP and ALP, ELISA experiments, bone resorption experiments, immunofluorescence (IF) and qRT-PCR experiments to detect the differentiation and maturation of osteoclasts and osteoblasts.

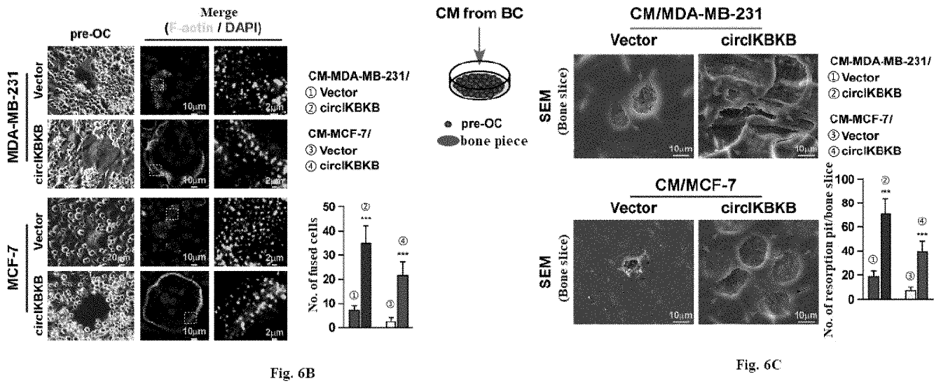

As shown in FIGS. 6A-6C, consistent with the in vivo experiments, in pre-osteoclast cells (pre-oc) treated with high-expressing circIKBKB breast cancer cells (CM-BC/circIKBKB) conditioned medium, the number of TRAP⁺ multinucleated mature osteoclasts and TRAP enzymatic activity was significantly increased (FIG. 6A). However, CM-BC/circIKBKB stimulation had no effect on the differentiation of pre-osteoblasts, and there was no significant change in the number of ALP⁺ osteoblasts (FIG. 6A). These results suggest that overexpression of circIKBKB in breast cancer cells induces osteoclastogenesis.

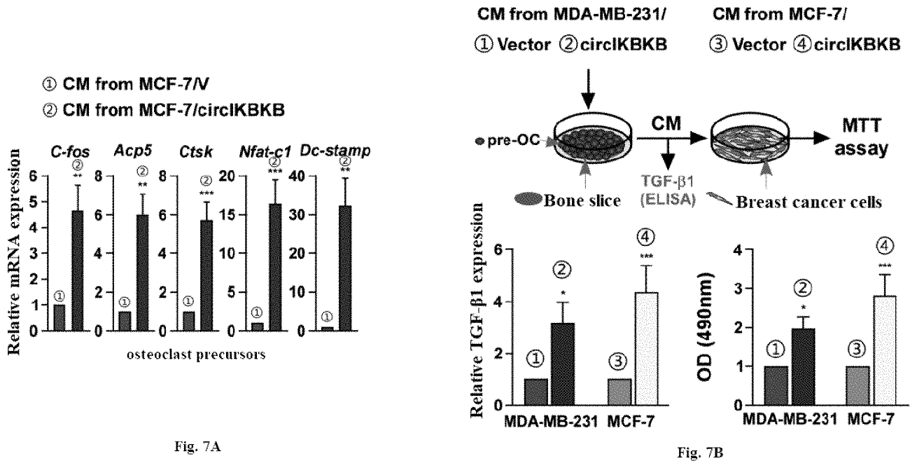

Combined with FIGS. 7A-7B, it can be seen that, in fact, CM-BC/circIKBKB treatment induced the expression of various osteoclastogenesis-related markers, including FBJ osteosarcoma oncogene (C-fos), acid phosphatase 5, tartrate resistance (Acp5), cathepsin K (Ctsk), activated T cell nuclear factor 1 (nfatc-c1), and dendritic cells express seven transmembrane proteins (Dc-stamp) that promote fusion of pre-osteoclast, along with the increasement of actin ring formation (as shown in FIG. 6B and FIG. 7A). Importantly, bone resorption assays revealed that CM-BC/circIKBKB-treated osteoclasts had higher bone resorption activity (FIG. 6C), resulting in increased levels of transforming growth factor β (TGF-(β) released from the bone matrix, thereby promoting breast cancer cells proliferated (FIG. 7B). Taken together, the results suggest that upregulation of circIKBKB in breast cancer cells can induce osteoclastogenesis.

Embodiment 2

An application of antisense oligonucleotides (ASOs) targeting the back-splice junction sequence of circIKBKB in the preparation of drugs for the treatment of breast cancer bone metastasis.

The ASOs sequences targeting the circIKBKB back-splice junction sequence are:

circIKBKB-ASO#1:
(SEQ ID NO: 4)
5′-GCTGAGTGACATTGGAAACAGGTG-3′
circIKBKB-ASO#2:
(SEQ ID NO: 5)
5′-GAGTGACATTGGAAACAGGTGAGC-3′

Experiment 1: SCP2 cells were seeded into the left ventricle of nude mice (1*106 cells/mouse), and mice were injected with 10 nmol ASOs through the tail vein twice a week. Mice were sacrificed after 60 days, and the hindlimbs were taken for μCT analysis, H&E and TRAP staining.

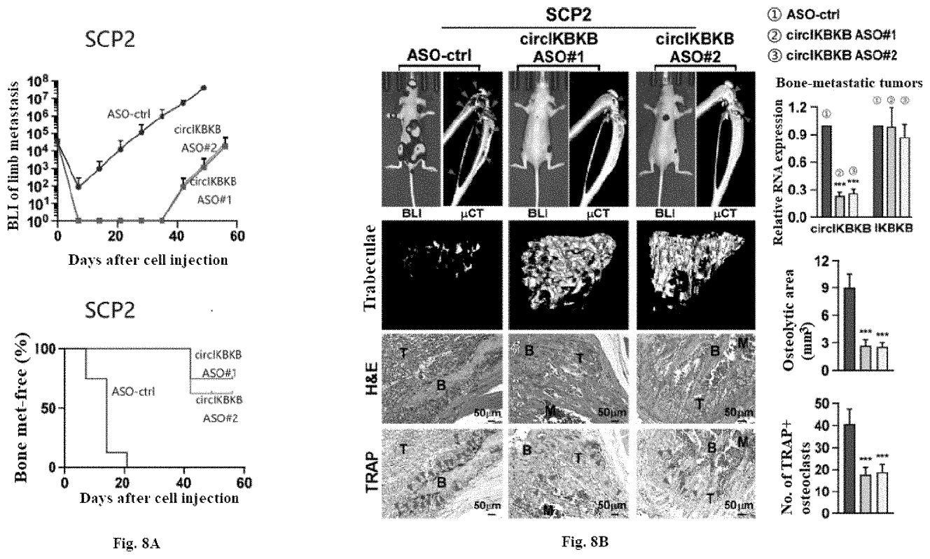

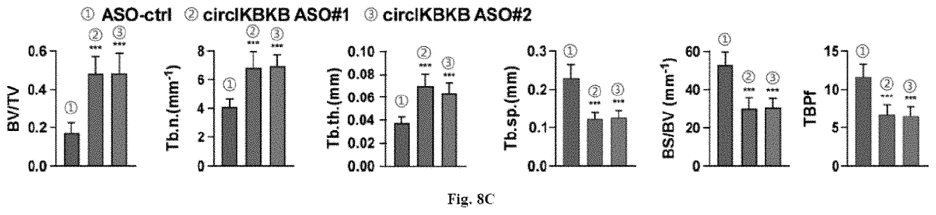

Compared with ASOs control-treated mice, circIKBKB-ASO-treated mice had significantly less and delayed bone metastasis, and significantly reduced the metastatic burden in SCP2 cell-injected mice (FIG. 8A and FIG. 8B).

μCT statistical analysis showed that circIKBKB-ASO-treated mice significantly reduced bone metastatic lesions/osteolytic areas, relatively increased trabecular volume/number/thickness, and decreased trabecular separation and bone pattern factors (FIG. 8C). Importantly, circIKBKB-ASO treatment significantly reduced TRAP+ osteoclasts at the bone-tumor interface compared with control mice (FIG. 8B).

Taken together, these results suggest that silencing of circIKBKB using ASOs targeting the back-splice junction sequence of circIKBKB can inhibit breast cancer bone metastasis in vivo.

Experiment 2: Osteoclast precursors were treated with the cell supernatant of SCP2 treated with ASOs (50 nM), and then the osteoclasts differentiation and maturation were detected by TRAP staining, ELISA experiments, bone resorption experiments, immunofluorescence (IF) and qRT-PCR experiments.

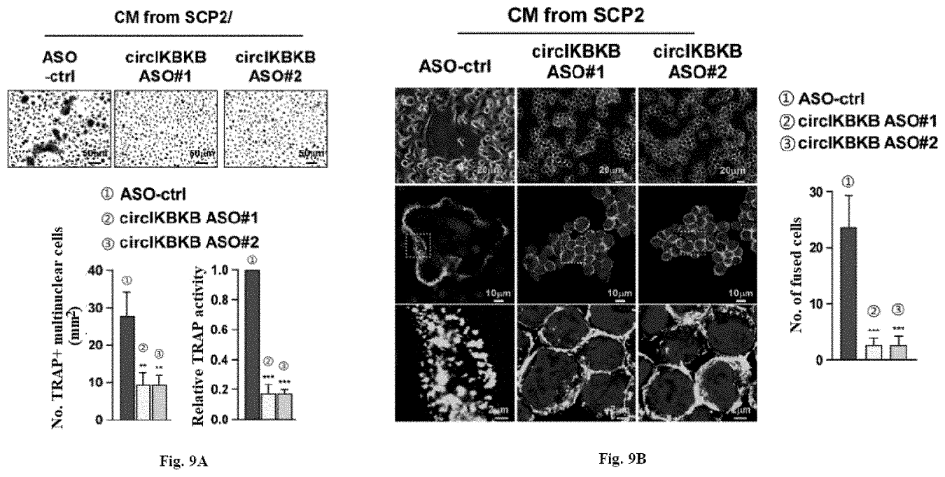

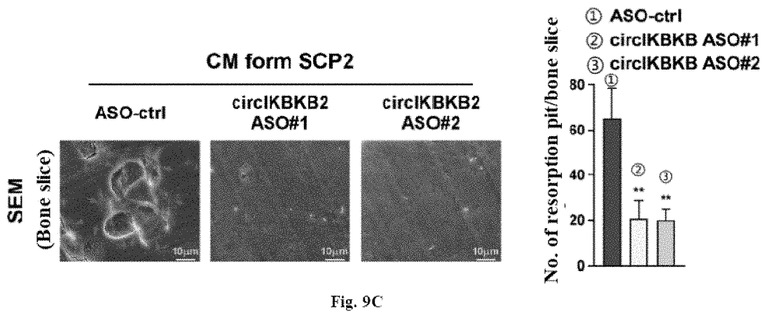

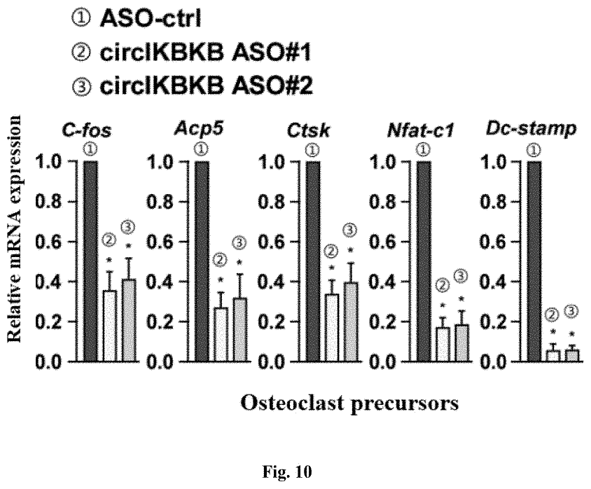

Silencing circIKBKB using ASOs targeting the back-splice junction sequence of circIKBKB significantly reduced the induction of osteoclastogenesis by CM/SCP2 cells. It was manifested as decreased TRAP⁺ multinucleated mature osteoclasts and TRAP enzyme activity, and decreased expression of osteoclastogenesis-related markers (FIG. 9A and FIG. 10). Correspondingly, downregulation of circIKBKB abrogated the stimulatory effects of CM/SCP2 on osteoclast fusion events and bone resorption activity (FIG. 9B and FIG. 9C). Meanwhile, the above results further proved that circIKBKB plays a crucial role in the induction of osteoclastogenesis in vitro.

In conclusion, the antisense oligonucleotides (ASOs) targeting the back-splice junction sequence of circIKBKB can be used to prepare drugs for the treatment of breast cancer bone metastasis.

Embodiment 3

An application of an inhibitor eIF4A3-IN-2 in the preparation of a drug for the treatment of breast cancer bone metastasis.

1. EIF4A3 is involved in the generation of circIKBKB

Experiment 1: In SCP2 cells, RNA pull down analysis was performed with circIKBKB pre-mRNA prepared by in vitro transcription, and mass spectrometry-based proteomic analysis was performed.

Experiment 2: RNAi system was used, and the effects of knockdown splicing factors PTBP1, EIF4A3 and FUS on circIKBKB expression were detected by qRT-PCR experiment.

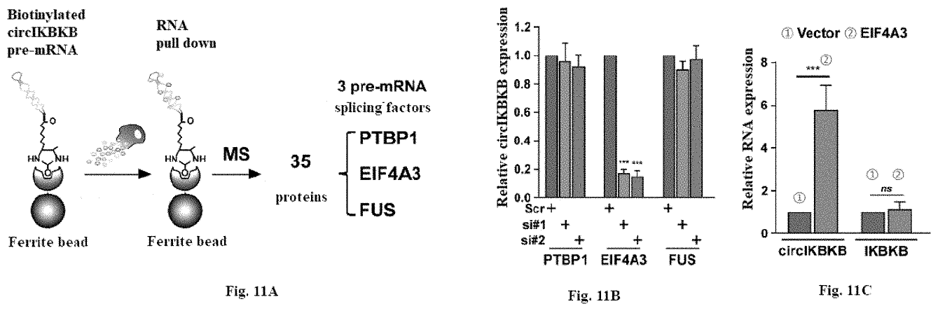

As shown in FIG. 11A, a total of 35 proteins were identified as potent circIKBKB pre-mRNA interacting proteins, including 3 pre-mRNA splicing factors, which were PTBP1, EIF4A3 and FUS.

Further qRT-PCR analysis found that silencing EIF4A3 significantly reduced the expression of circIKBKB in breast cancer cells, while overexpression of EIF4A3 increased the expression of circIKBKB (FIG. 11B and FIG. 11C).

Overexpression of EIF4A3 did not affect the expression level of circIKBKB's parental gene, IKBKB (FIG. 11C), suggesting that EIF4A3 may be involved in the cyclization of circIKBKB.

Experiment 3: Analyzed through the Circinteractome (https://circinteractome.nia.nih.gov/index.html) website, and used RNA-binding protein immunoprecipitation (RIP) experiments and RNA pull-down experiments to verify the binding site of EIF4A3 in circIKBKB pre-mRNA.

As shown in FIG. 12A, Circinteractome website analysis showed that EIF4A3 has 8 putative binding sites in the upstream and downstream regions of circIKBKB pre-mRNA.

RIP analysis revealed that EIF4A3 was only associated with putative binding sites near exon 3 and exon 5 in circIKBKB pre-mRNA (FIG. 12A).

These results were confirmed by RNA pull-down experiments using in vitro circIKBKB pre-mRNA transcript fragments (FIG. 12B).

Therefore, the above results suggest that EIF4A3 directly binds to circIKBKB pre-mRNA and induces circIKBKB cyclization.

2. EIF4A3 affects osteoclast differentiation induced by breast cancer cell lines through circIKBKB.

Experiment 1: Osteoclast precursors were treated with supernatant of breast cancer cells with high expression of EIF4A3 or knockdown of circIKBKB first and then replenishment of EIF4A3 expression, and then the differentiation and maturation of osteoclasts was detected by TRAP staining and ELISA experiments.

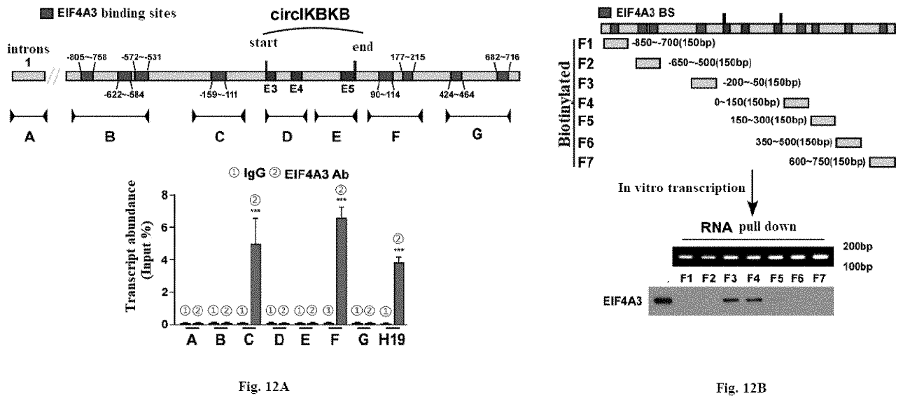

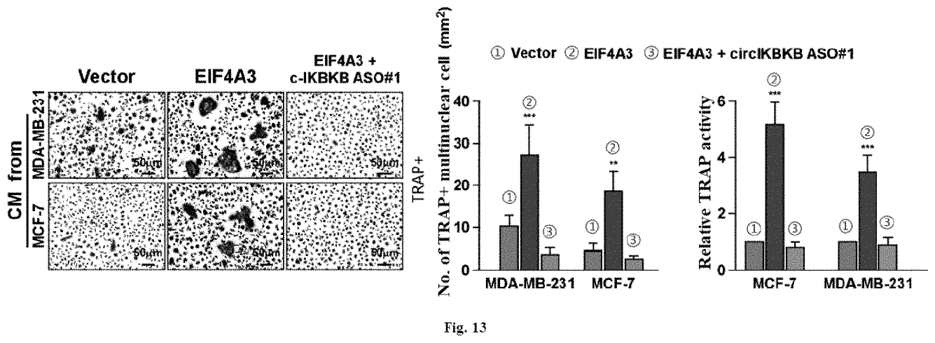

As shown in FIG. 13, high expression of EIF4A3 significantly increased the induction of osteoclastogenesis in breast cancer cells, manifested as increased TRAP⁺ multinucleated mature osteoclasts and TRAP enzyme activity, and knockdown of circIKBKB could reverse this effect of EIF4A3, suggesting that EIF4A3 affects breast cancer cell line-induced osteoclast differentiation through circIKBKB.

3. EIF4A3 overexpression is clinically associated with bone-metastatic breast cancer.

Experiment 1: The expression of EIF4A3 was detected in 20 normal breast tissues and 331 clinical breast cancer tissues were tested by immunohistochemistry (IHC), including 295 primary breast cancer tissues (237 without bone metastasis, 58 with bone metastasis), 36 bone-metastatic breast cancer tissues (at bone).

Experiment 2: In 58 primary breast cancer tissues with bone metastasis, the samples were divided into two groups with high expression of EIF4A3 and low expression of EIF4A3 according to the experimental results of IHC, and the correlation between the two groups of samples and breast cancer bone metastasis was analyzed.

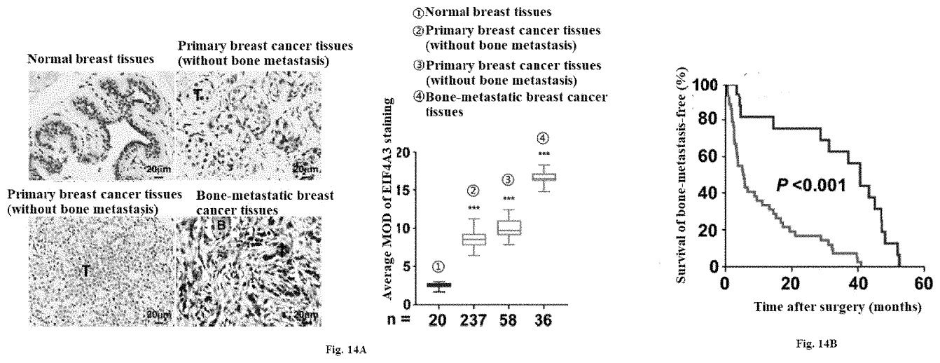

As shown in FIGS. 14A-14B, compared with normal breast tissues, EIF4A3 expression was moderately elevated in breast cancer tissues (without bone metastasis) and primary breast cancer tissues (with bone metastasis), while the expression is strongly elevated in bone-metastatic breast cancer tissues. Importantly, compared with breast cancer patients with low EIF4A3 expression, the bone-metastasis-free survival rate of breast cancer patients with high EIF4A3 expression was significantly shorter (P<0.001). These results suggest that the overexpression of EIF4A3 is clinically associated with the breast cancer bone metastasis.

Experiment 3: The expression levels of EIF4A3 and circIKBKB were detected by IHC and ISH experiments in 36 bone metastatic breast cancer tissues, and the expression correlation of EIF4A3 and circIKBKB was statistically analyzed.

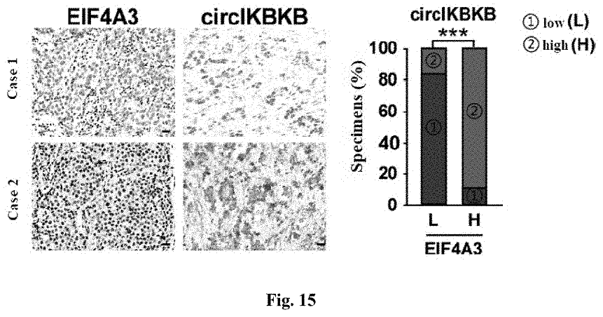

As shown in FIG. 15, statistical analysis showed that the expression level of EIF4A3 was positively correlated with the expression level of circIKBKB (P<0.001).

4. EIF4A3 inhibitor eIF4A3-IN-2 not only prevents the initiation of breast cancer bone metastasis but also suppresses the progression of breast cancer to bone metastasis.

Experiment 1: (1) Osteoclast precursors were treated with supernatants of breast cancer cells with EIF4A3 knockdown or the inhibitor of EIF4A3, eIF4A3-IN-2, and the differentiation and maturation of osteoclasts were detected by TRAP staining, ELISA and bone absorption assay.

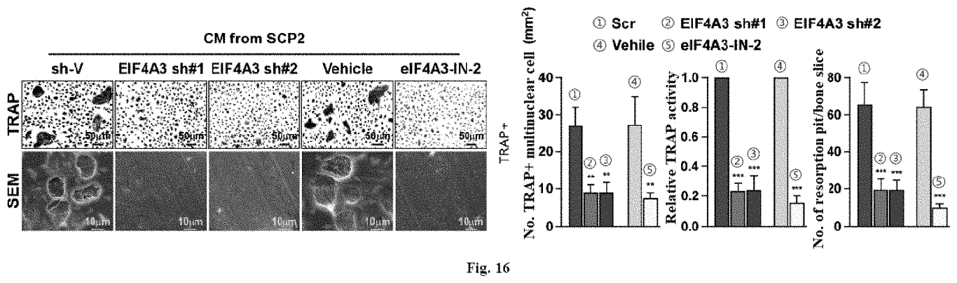

As shown in FIG. 16, EIF4A3 inhibition also significantly reduced the ability of breast cancer cells to induce osteoclastogenesis, as demonstrated by reduced numbers of TRAP⁺ multinucleated osteoclasts and TRAP activity, and reduced bone resorption activity.

Experiment 2: SCP2 cells were injected into the left ventricle of nude mice (1*106 cells/mouse), and eIF4A3-IN-2 (1 mg/kg) was administered to the mice by intragastric administration from the second day. The mice were sacrificed after 60 days, and hindlimbs of the mice were taken for μCT analysis, H&E and TRAP staining.

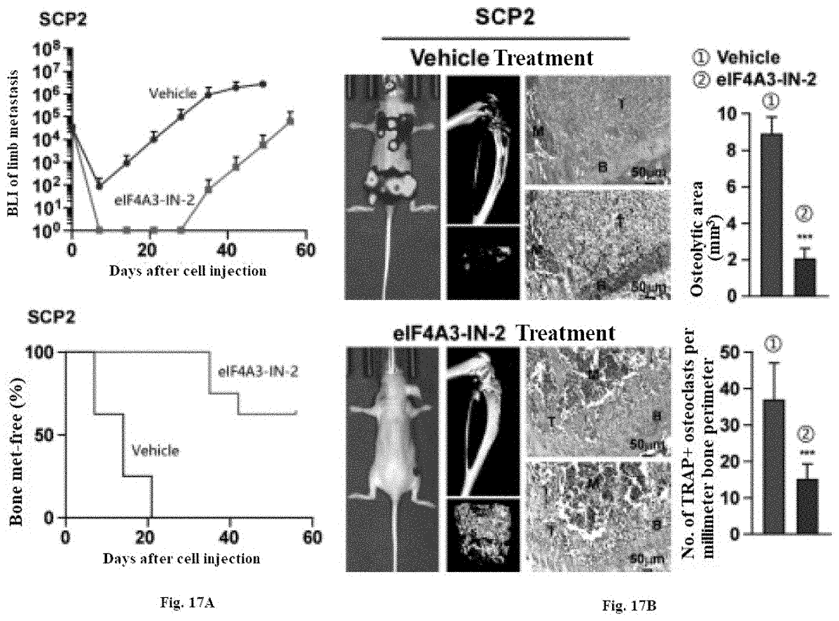

As shown in FIGS. 17A-17B, compared to vehicle treatment, eIF4A3-IN-2 treatment significantly delayed the onsets of bone-metastases and bone-metastasis burden.

The eIF4A3-IN-2-treated mice displayed less osteolytic areas and number of TRAP+-osteoclasts in bone surface area (FIGS. 17A and 17B). These results suggest that eIF4A3-IN-2 treatment may prevent breast cancer bone metastasis.

Experiment 3: SCP2 cells were injected into the left ventricle of nude mice (1*106 cells/mouse). eIF4A3-IN-2 (1 mg/kg) treatment via intragastric administration was started when bioluminescence signal of bone-metastatic tumors reached 2×10⁷ p/sec/cm²/sr. The mice were sacrificed after 60 days, and the hindlimbs of the mice were taken for μCT analysis, H&E and TRAP staining.

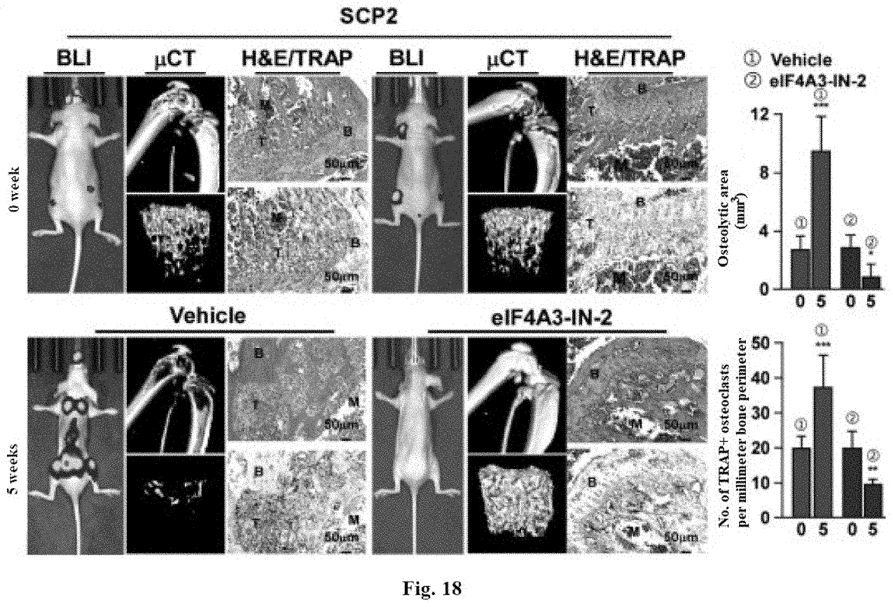

As shown in FIG. 18, After 5 weeks treatment, the vehicle-treated mice exhibited rapid progress of bone metastasis, which showed more bone metastases and larger bone-metastatic tumor burden, accompanying with severe osteolytic bone lesions and higher numbers of TRAP+-osteoclasts along the bone-tumor interface;

Strikingly, eIF4A3-IN-2 treatment dramatically reduced the onsets of bone metastases and decreased bone-metastatic tumor burden compared to vehicle treatment. Therefore, the above results suggest that pharmaceutical inhibition of EIF4A3 not only prevents the initiation of breast cancer bone metastasis, but also suppresses the progression of breast cancer to bone metastasis.

Accordingly, the results show that the eIF4A3-IN-2 can be used in the preparation of drugs for the treatment of breast cancer bone metastasis.

What is claimed is:

1. An application of a testing reagent for expression level of molecular marker circIKBKB in the preparation of a diagnosis, treatment and prognosis reagent for breast cancer bone metastasis.

2. The application of the testing reagent for expression level of molecular marker circIKBKB in the preparation of the diagnosis, treatment and prognosis reagent for breast cancer bone metastasis according to claim 1, wherein the circular RNA marker circIKBKB is has_circ_0084100.

3. The application of the testing reagent for expression level of molecular marker circIKBKB in the preparation of the diagnosis, treatment and prognosis reagent for breast cancer-bone metastasis according to claim 1, wherein the breast cancer includes one of ER⁺ breast cancer, HER2⁺ breast cancer, and triple negative breast cancer.

4. The application of a testing reagent for the expression level of molecular marker circIKBKB in the diagnosis, treatment and prognosis kit of breast cancer bone metastasis according to claim 1.

5. A kit for detecting expression level of molecular marker circIKBKB, wherein the kit comprises a reagent capable of quantitatively detecting the expression level of circIKBKB;
the kit comprises RT-PCR, Q-PCR, Northern blot, FISH or ISH kits.

6. The kit for detecting expression level of molecular marker circIKBKB according to claim 5, wherein the kit comprises primers shown in SEQ ID NOs: 1-2 in the sequence listing or a probe shown in SEQ ID NO: 3 in the sequence listing for the linker sequence of circIKBKB.

7. A reagent and a medicine for treating breast cancer bone metastasis which using eIF4A3-IN-2 as an inhibitor to inhibiting the generation of circIKBKB and for preparing a drug for treating breast cancer bone metastasis.


SEQUENCE LISTING (5 sequences)

SEQ ID NO: 1 - DNA, 21 bp, synthetic construct
  source 1..21: mol_type = other DNA
ccagtttgagaactgctgtgg

SEQ ID NO: 2 - DNA, 19 bp, synthetic construct
  source 1..19: mol_type = other DNA
cggcactgcttgatggcaa

SEQ ID NO: 3 - DNA, 50 bp, synthetic construct
  source 1..50: mol_type = other DNA
atcctcaccttgctgagtgacattggaaacaggtgagcagattgccatca

SEQ ID NO: 4 - DNA, 24 bp, synthetic construct
  source 1..24: mol_type = other DNA
gctgagtgacattggaaacaggtg

SEQ ID NO: 5 - DNA, 24 bp, synthetic construct
  source 1..24: mol_type = other DNA
gagtgacattggaaacaggtgagc